(19) United States
(12) Patent Application Publication
(10) Pub. No.: US 2026/0363965 A1
(43) Pub. Date: Nov. 19, 2026

(54) Oral Wrap Whitening System Featuring Adhesive-Free Conformal Delivery and Botanical-Enzymatic Whitening Composition

(71) Applicants: Dakota Morse, Venice, CA (US); J. Henry Scott, Boca Raton, FL (US)

(72) Inventors: Dakota Morse, Venice, CA (US); J. Henry Scott, Boca Raton, FL (US)

(21) Appl. No.: 19/208,599

(22) Filed: May 15, 2025

(51) Int. Cl. A61K 8/02; A61K 8/66; A61K 8/92; A61K 8/9789; A61K 8/9794; A61Q 11/00

(52) U.S. Cl. CPC A61K 8/0208; A61K 8/66; A61K 8/922; A61K 8/9789; A61K 8/9794; A61Q 11/00; A61K 2800/30

(57) ABSTRACT

An oral whitening wrap system is disclosed, comprising a conformable, biocompatible substrate configured to envelop one or more dental arches and a whitening composition disposed thereon. The composition includes naturally derived ingredients such as papain, bromelain, coconut oil, aloe vera extract, and green tea extract, and is substantially free of peroxide-based agents. The wrap adheres to the teeth without the use of synthetic adhesives, utilizing a saliva-activated hydration mechanism that enables secure, reversible contact with dental surfaces. The system delivers uniform whitening across labial, lingual, and interproximal regions and is suitable for daily, at-home use. The wrap is fabricated from hydrophilic polymers and may be configured for single or dual-arch application, offering an ergonomic, sensitivity-free alternative to conventional whitening products.

### BACKGROUND OF THE INVENTION

### Field of Invention

The present invention relates generally to the field of dental hygiene and cosmetic oral care, and more particularly to tooth whitening systems. Specifically, the invention pertains to a conformable oral wrap designed to deliver a whitening composition to dental surfaces without the use of adhesives. The invention further relates to an adhesive-free, dual-arch delivery mechanism capable of uniformly applying botanical and enzymatic whitening agents to both upper and lower teeth surfaces simultaneously, providing improved coverage, user comfort, and sensitivity-free application in an at-home whitening regimen. ds

### BRIEF SUMMARY OF THE INVENTION

The present invention provides a novel oral whitening wrap system comprising a flexible, conformable substrate configured to envelop both upper and lower dental arches simultaneously or individually, depending on the embodiment. The wrap delivers a peroxide-free whitening composition containing natural enzymatic and botanical ingredients that remove extrinsic stains while minimizing enamel erosion and tooth sensitivity. The whitening agents are evenly distributed across the interior-facing surface of the wrap, which maintains uniform contact with teeth through a saliva-activated adherence mechanism, eliminating the need for synthetic adhesives or mechanical clips.

The oral wrap is fabricated from a biocompatible, hydrophilic material that adapts to dental topography and resists displacement during the treatment period. The design allows for complete and simultaneous whitening coverage across multiple teeth surfaces, including labial, lingual, and interproximal regions, improving overall efficacy compared to conventional strips or trays. The invention further supports user-friendly, at-home application with simplified placement and removal procedures, offering a safe and effective alternative to peroxide-based whitening systems.

### BRIEF DESCRIPTION OF THE FIGURES

FIG. 1 illustrates a top-down view of the oral whitening wrap in its pre-application state, showing the exterior-facing surface and overall geometric configuration.

FIG. 2 shows a bottom-up view of the oral whitening wrap, revealing the interior-facing surface that carries the active whitening composition.

### DETAILED DESCRIPTION

The present invention provides a conformable, intraoral whitening wrap configured to envelop dental structures in a flexible and anatomically adaptive manner. Unlike conventional whitening strips or molded trays that rely on rigid or semi-rigid forms, the oral wrap disclosed herein is fabricated from a soft, pliable substrate capable of folding, wrapping, or curving around both labial (front-facing) and lingual (tongue-facing) surfaces of the teeth. This architecture enables complete three-dimensional coverage of the dental arches and ensures simultaneous exposure of multiple tooth surfaces to the active whitening composition.

The oral wrap comprises an elongated, ribbon-like structure with an engineered geometry that permits application to either the maxillary (upper) or mandibular (lower) arch, or in certain embodiments, to both arches simultaneously. The wrap may be fabricated in a bifurcated or symmetrical format, with a medial portion (101) adapted to engage the anterior teeth and two extending lateral arms (103, 105) that conform to the premolar and molar regions. The wrap may be pre-folded, crease-marked, or pre-molded to facilitate ease of application and ensure secure placement during wear. A curvature memory feature may also be imparted to the material during fabrication to allow the wrap to retain a partially preformed shape that naturally hugs the dental contours upon placement.

The substrate material from which the oral wrap is constructed is selected for its combination of biocompatibility, elasticity, moisture responsiveness, and conformal integrity. Preferred materials include hydrophilic polymers such as hydroxypropyl cellulose (HPC), hydroxyethyl cellulose (HEC), or polyvinyl alcohol (PVA), optionally reinforced with plant-derived fibers or biodegradable mesh to enhance mechanical stability. The material possesses sufficient flexibility to allow dynamic movement of the jaw and tongue during the whitening period, without detachment or user discomfort. The thickness of the wrap ranges from approximately 100 to 500 microns, with variable thickness zones to allow increased flexibility in interproximal areas while retaining mechanical strength along the occlusal and incisal margins.

The wrap may further incorporate a multilayered construction, comprising at least (i) a composition-carrying layer, and optionally, (ii) a barrier or retention layer. The composition-carrying layer (107) serves as the reservoir for the whitening formulation and is positioned on the interior-facing surface that contacts the dentition. In certain embodiments, this layer includes micro-reservoirs or printed microdots of the formulation to provide targeted, controlled release. The barrier layer (109), if present, may be composed of a hydrophobic or semi-occlusive material such as ethylene-vinyl acetate (EVA) or biodegradable wax to prevent evaporation of moisture and enzymatic degradation before use. The layers may be laminated, co-extruded, or thermo-bonded during fabrication.

To enhance user experience and compliance, the oral wrap may be configured with ergonomic features such as tapering ends (111), arch curvature profiles, and tactile orientation cues (e.g., textured grip zones or indicators for top/bottom placement). In some embodiments, a scored or perforated central region may allow the wrap to be easily split for individual arch application. The wrap may also exhibit bidirectional flexibility to accommodate movement across both sagittal and transverse dental axes, improving adaptation to varying oral anatomies.

The invention further contemplates multiple size variants to account for anatomical variability among users. Pediatric, small, medium, and large adult sizes may be produced, with each conforming to standardized dental arch dimensions. The wrap's flexibility permits intraoral adjustments without the need for trimming, thereby reducing error and maximizing surface contact. The design eliminates the need for impression molding, custom trays, or dental supervision, rendering the product suitable for over-the-counter consumer distribution.

Collectively, the oral wrap architecture of the present invention constitutes a significant departure from rigid and strip-based whitening devices, offering a flexible, comfortable, and full-coverage system that is compatible with peroxide-free botanical whitening technologies. The conformable geometry enhances uniformity of whitening results, user safety, and application convenience across a wide range of users and oral anatomies.

The present invention utilizes a peroxide-free, biologically derived whitening composition embedded within the oral wrap, formulated to deliver safe and effective removal of extrinsic stains from dental enamel without the deleterious effects typically associated with conventional bleaching agents. The composition employs a synergistic blend of enzymatic, botanical, and biocompatible constituents, each selected for their oral safety, whitening efficacy, and ability to function optimally in a moist, intraoral environment.

Central to the formulation is a proteolytic enzyme complex comprising papain and bromelain—enzymes derived respectively from Carica papaya and Ananas comosus. These enzymes catalyze the hydrolysis of peptide bonds within chromogenic proteinaceous materials adhered to tooth surfaces. Unlike peroxide-based oxidizing systems that exert broad-spectrum chemical degradation, the enzymatic mechanism employed herein selectively targets extrinsic stains without compromising the mineral integrity of the underlying enamel. Papain and bromelain are used in combined concentrations ranging from approximately 0.1% to 2.5% w/w, balanced to ensure effective substrate cleavage while minimizing potential mucosal irritation.

The whitening composition further incorporates coconut oil (Cocos nucifera) and aloe vera extract (Aloe barbadensis), both of which act as natural humectants and emollients, contributing to oral tissue hydration and comfort during the whitening process. Coconut oil, rich in medium-chain fatty acids such as lauric acid, provides ancillary antimicrobial properties and enhances formulation spreadability across enamel surfaces. Aloe vera extract introduces polysaccharides and bioactive compounds including acemannan, which confer anti-inflammatory and epithelial-protective effects. These botanical agents collectively aid in the reduction of post-treatment sensitivity and promote mucosal tolerance.

To reinforce the antimicrobial and antioxidative profile of the composition, green tea extract (Camellia sinensis) is included in concentrations ranging from approximately 0.1% to 3.0% w/w. The extract contains high levels of catechins, including epigallocatechin gallate (EGCG), which have been demonstrated to inhibit bacterial biofilm formation and support gingival health. In the context of whitening, these effects help to prevent stain recurrence and support a cleaner oral environment during and after treatment.

The formulation optionally includes a buffering system comprising sodium citrate or potassium bicarbonate to maintain a physiologically compatible pH between 6.0 and 7.5. This buffering range ensures enzyme stability and activity while preventing enamel demineralization or hypersensitivity associated with acidic conditions. In preferred embodiments, the pH is further optimized to support the natural oral microbiome and minimize disruption to salivary homeostasis.

As delivery within the oral wrap requires uniform dispersion and retention during application, the composition includes rheology modifiers such as xanthan gum, hydroxyethyl cellulose, or carrageenan in concentrations sufficient to impart viscosity without interfering with active agent bioavailability. These agents stabilize the formulation on the wrap substrate and assist in controlling the release rate upon contact with saliva.

The composition is substantially free of hydrogen peroxide, carbamide peroxide, peracetic acid, and related oxidants, and contains no synthetic bleaching agents or alcohol-based solvents. The use of exclusively natural or naturally derived active agents enables the product to be marketed as a clean-label or “natural oral care” solution, suitable for users with chemical sensitivities, dental restorations, or concerns regarding enamel erosion.

Furthermore, the whitening composition is designed for long-term shelf stability. In its dry or semi-solid state embedded within the oral wrap, the formulation remains inactive until hydrated by saliva during use. Packaging under low-humidity, oxygen-barrier conditions ensures enzymatic activity is preserved for at least 18 months under standard storage conditions, in accordance with ICH guidelines for cosmetic and therapeutic products.

In sum, the whitening composition employed in the present invention offers a biocompatible, peroxide-free alternative to conventional dental whitening products, formulated for safe intraoral delivery via a flexible wrap system. The integration of enzymatic and botanical agents supports effective stain removal, user comfort, and mucosal safety, while enabling over-the-counter deployment in consumer-facing oral care markets.

The present invention discloses a novel, adhesive-free delivery system in which the oral whitening wrap adheres securely to dental surfaces through a saliva-activated mechanism. This feature obviates the need for traditional pressure-sensitive adhesives or thermoplastic fitting systems, which are often associated with mucosal irritation, user discomfort, and manufacturing complexity. Instead, the present invention leverages the physicochemical properties of hydrophilic polymer substrates and the natural moisture content of the oral environment to enable stable, atraumatic adherence during the whitening procedure.

Upon introduction into the oral cavity, the hydrophilic components of the wrap substrate undergo rapid hydration in response to ambient saliva. The polymers—preferably hydroxypropyl cellulose (HPC), polyvinyl alcohol (PVA), or similar bioadhesive agents—absorb moisture through diffusion and capillary action, triggering polymer chain relaxation, swelling, and softening. This hydration-induced transformation results in a conformal engagement of the wrap material with the teeth and surrounding oral anatomy, including interdental and gingival contours. The interaction is primarily driven by hydrogen bonding, Van der Waals forces, and physical interlocking, providing a secure, reversible bond without the introduction of synthetic adhesive compounds.

The system is further characterized by its self-adapting behavior: as the material continues to hydrate over a defined initial window (typically 15-60 seconds), its viscoelasticity increases, enabling the wrap to drape naturally over labial, lingual, and occlusal surfaces without requiring manual pressure or mold-based shaping. The hydrophilic matrix acts as both a conformal contact layer and a controlled release platform for the whitening composition. Once fully hydrated, the wrap maintains positional stability for a continuous treatment period ranging from 10 to 45 minutes, depending on user preference and formulation design.

To enhance the efficacy of this adhesion mechanism, the wrap may incorporate engineered surface features on the interior-facing layer, such as microtexturing, embossing, or microchanneling. These features increase the effective surface area and promote capillary retention of saliva, thereby stabilizing the hydration layer and preventing slippage. In some embodiments, discrete zones of increased porosity or hydrophilic loading may be positioned along the central and lateral segments of the wrap to anchor critical contact points against the dentition.

The system is designed to maintain mechanical and structural integrity throughout the wear period. The material resists premature delamination or sloughing under dynamic oral conditions, such as jaw movement, speech, and salivary flow. At the conclusion of the whitening interval, the wrap can be removed in one piece by manual extraction or will naturally detach as hydration reaches a critical threshold that exceeds the material's cohesive strength. This reversibility ensures that no adhesive residue or formulation by-product remains on the enamel, gums, or mucosa, thereby eliminating the need for post-use brushing, rinsing, or scraping.

An additional advantage of the saliva-activated mechanism is its compatibility with a wide range of users, including those with restorations, orthodontic appliances, or hypersensitive tissues. By avoiding chemically aggressive adhesives and minimizing pressure-based application, the wrap reduces the risk of irritation, soft tissue abrasion, and user error. The system is also inherently scalable, allowing for adaptation to various wrap geometries, sizes, and formulation profiles without altering the fundamental adhesion principle.

Moreover, the saliva-activated delivery mechanism contributes to product stability by isolating the activation trigger (saliva) from the whitening composition during storage. This separation prevents premature hydration or enzymatic degradation and extends the shelf-life of the embedded actives. During manufacturing, the absence of adhesives simplifies the production line, reduces material costs, and supports compliance with clean-label or natural product standards sought by regulatory agencies and consumers.

In summary, the saliva-activated, adhesive-free delivery and retention mechanism of the present invention provides a biocompatible, ergonomic, and technically superior method of securing a whitening composition to dental surfaces. It enhances both functional performance and user experience while addressing long-standing deficiencies in prior art whitening systems.

The oral whitening wrap of the present invention is engineered for simplified, intuitive application by the end user, without the need for professional dental supervision, custom fitting, or specialized tools. The application methodology is designed to maximize user compliance, comfort, and whitening efficacy, and supports both single-arch and dual-arch treatment protocols. The device may be self-administered as part of a cosmetic dental hygiene routine, either in isolation or as a component of a broader oral care regimen.

To initiate treatment, the user removes the individually packaged oral wrap from a sterile, moisture-impermeable pouch. Upon removal, the wrap remains dry, pliable, and pre-loaded with the whitening composition. The user may then position the wrap over the maxillary and/or mandibular arch by gently placing it against the front teeth and wrapping the lateral portions around the molars and lingual surfaces. The wrap's geometry, optionally including anatomical curvature, pre-folded creases, or size-specific tailoring, allows it to conform to the dental arch without the need for trimming, heating, or pressure fitting.

Upon intraoral placement, ambient saliva hydrates the wrap substrate and activates the adhesive-free retention system as previously described in Section 3. This hydration process enables the wrap to securely affix to enamel surfaces while releasing the whitening formulation in a controlled, sustained manner. The wrap is designed to provide comprehensive coverage across multiple dental surfaces, including labial, lingual, and interproximal zones, thereby enhancing uniformity of whitening and reducing the occurrence of uneven shade gradients or untreated areas commonly associated with strip-based systems.

The recommended duration of application ranges from approximately 15 to 30 minutes per session, with treatment frequency suggested at once or twice daily for a period of 7 to 14 days, depending on the degree of discoloration and the user's aesthetic goals. The duration and frequency are optimized to enable gradual whitening results while minimizing irritation or sensitivity. Due to the absence of peroxide and other oxidizing agents, the wrap may be used more frequently than conventional treatments, and is suitable for users with prior whitening sensitivity or restorations such as veneers, crowns, or bonded composites.

The invention supports both single-arch and simultaneous dual-arch application. In a dual-arch protocol, two wraps—one for each arch—may be applied concurrently, streamlining the treatment process and reducing total time commitment. Alternatively, a single wrap may be applied to one arch at a time for sequential treatment. In some embodiments, a unitary dual-arch wrap configuration may be employed, wherein a single device spans both the upper and lower teeth, connected at the posterior or by a central hinge, allowing for synchronized engagement. Such configurations are particularly suitable for at-home users seeking efficiency without compromising coverage.

Upon completion of the whitening interval, the user removes the wrap by gently peeling it away from the teeth. The hydrated substrate is designed to remain intact and remove cleanly, without leaving residue or requiring additional oral rinsing or brushing. However, users may optionally follow removal with a rinse or brushing step for enhanced freshness or comfort. The wrap is intended for single use and should be discarded after removal.

To support consumer use and ensure proper application, the product may be accompanied by printed instructions, pictorial diagrams, or QR code links to instructional videos. Optional compliance features include a color change indicator integrated into the wrap or packaging to signify correct hydration or usage duration. In some embodiments, flavoring agents such as mint, citrus, or neutral botanical flavors may be incorporated to improve palatability during treatment.

From a regulatory and commercial perspective, the application method qualifies the invention for classification as an over-the-counter cosmetic or therapeutic product, subject to labeling and formulation regulations depending on jurisdiction. The ease of use, comfort, and perceived naturalness of the system support consumer confidence and brand differentiation in highly competitive oral care markets.

In summary, the application methodology of the present invention provides an ergonomically optimized, peroxide-free whitening experience designed for daily consumer use. Its ease of deployment, compatibility with dual-arch treatment, and self-activating retention mechanism distinguish it from prior art whitening devices and support widespread adoption in consumer oral hygiene routines.

The manufacturing process of the oral whitening wrap described in the present invention integrates precision formulation handling, substrate engineering, and controlled environment packaging to ensure product efficacy, safety, and commercial scalability. The production methodology adheres to industry standards for oral care and cosmetic products, including Good Manufacturing Practices (GMP) and applicable ISO standards, such as ISO 22716 for cosmetics and ISO 13485 for medical devices where applicable.

The wrap substrate is fabricated using solvent casting, extrusion, or film lamination techniques, depending on the physical and chemical characteristics of the selected polymer materials. In a preferred embodiment, a hydrophilic polymer matrix—comprising hydroxypropyl cellulose (HPC), polyvinyl alcohol (PVA), or other biocompatible agents—is dissolved or suspended in an aqueous or hydroalcoholic solution. The resulting film-forming mixture is cast onto a flat carrier surface using slot-die or knife-over-roll coating methods, then passed through a controlled drying tunnel at temperatures ranging from 25 °C to 45 °C. Drying conditions are carefully regulated to prevent degradation of enzymatic actives while allowing complete solvent removal.

The whitening composition, consisting of botanical and enzymatic actives such as papain, bromelain, aloe vera extract, green tea extract, and coconut oil, is prepared separately under low-shear mixing conditions to preserve enzymatic bioactivity. Temperature-sensitive components are handled in climate-controlled environments, with mixing temperatures not exceeding 35 °C. Once formulated, the composition is applied to the wrap substrate using a variety of deposition methods including screen printing, gravure coating, or micro-reservoir dosing to ensure even and reproducible distribution. In multilayer embodiments, the whitening formulation may be deposited onto a specific layer and laminated with an outer barrier film to form a bilaminar or trilaminar wrap configuration.

Following deposition, the wraps are die-cut into individual units using rotary or flatbed die-cutting systems. Shapes and dimensions are determined by ergonomic and anatomical design criteria, including curvature, wrap length, and target dentition coverage. Each wrap is then inspected for visual integrity, uniformity of composition loading, and dimensional consistency under high-resolution imaging or machine-vision systems. Quality assurance processes include enzymatic activity assays, pH verification, microbial load testing, and tensile property evaluation to ensure compliance with release specifications.

Packaging is performed under low-humidity, contaminant-controlled conditions to preserve the stability of the composition. Each wrap is sealed within an individual pouch composed of multilayered barrier film—typically polyethylene terephthalate (PET)/aluminum foil/polyethylene laminates—that provides moisture, oxygen, and light protection. Where required, inert gas flushing (e.g., nitrogen) or desiccant inserts may be used to further limit oxidative degradation and enzymatic inactivation. The packaging may incorporate tamper-evident features, colorimetric freshness indicators, or QR codes for tracking and consumer education.

Stability testing is conducted in accordance with International Council for Harmonisation (ICH) guidelines, using both accelerated (40 °C, 75% RH) and real-time (25 °C, 60% RH) storage conditions. Over an 18-month period, the wraps demonstrate less than 10% loss in enzymatic activity, with no phase separation, microbial growth, or pH drift observed. Packaging materials are further tested for extractables and leachables under simulated use conditions to ensure biocompatibility and regulatory compliance.

In addition to maintaining compositional stability, the manufacturing process is designed for scalability and cost efficiency. Line speeds of up to 30 meters per minute are achievable using roll-to-roll systems, with batch sizes adaptable for pilot, mid-scale, or commercial production. Modular design allows for integration of additional features such as dual-compartment packaging (e.g., separate upper/lower arch wraps), printed dosing indicators, and inclusion of post-use oral rinse sachets.

The materials used throughout the manufacturing process are selected for their oral safety, biodegradability, and regulatory acceptance in target markets. All ingredients and packaging components comply with USP <61>/<62> microbial limits, FDA Inactive Ingredient Database (IID) entries (where applicable), and EU Cosmetic Regulation (EC) No. 1223/2009, depending on the classification of the final product.

In conclusion, the manufacturing and packaging methodology of the present invention ensures consistent quality, enzymatic stability, and long shelf life of the oral whitening wrap. The production techniques support large-scale commercial deployment while maintaining the formulation's natural, peroxide-free integrity, thereby reinforcing the product's suitability for the global consumer oral care market.

### DETAILED DESCRIPTION OF FIGURES

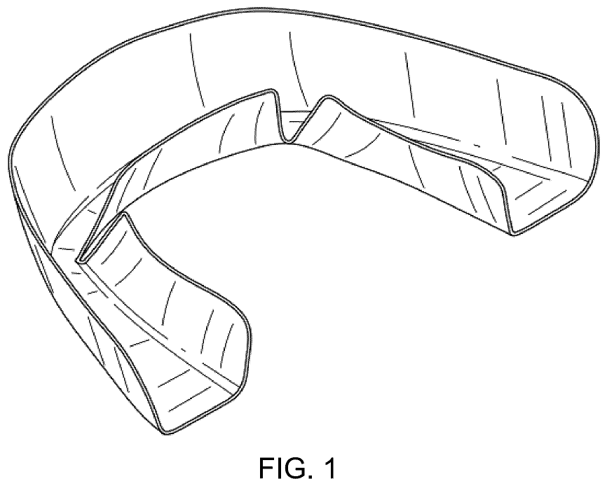

FIG. 1 depicts a planar, top-down view of the oral whitening wrap (101) as viewed from the exterior-facing side, i.e., the side not intended to contact the dental surfaces during use. The wrap comprises a central anterior segment (103) designed to align with the user's incisors and canines, and two lateral extension arms (105, 107) configured to wrap around the premolar and molar regions of the dental arch. The geometry of the wrap may exhibit anatomical curvature or tapering (109) to accommodate variations in arch width and tooth positioning. Orientation indicators (111), such as markings, textural features, or alignment cues, may be optionally printed or embossed on the top-facing surface to assist in proper placement by the user. In some embodiments, the top layer may include a barrier or occlusive laminate to protect the interior formulation from moisture ingress prior to use.

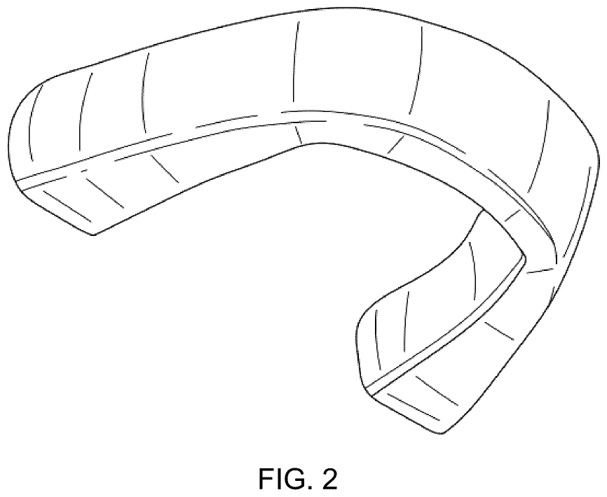

FIG. 2 illustrates a bottom-up view of the oral whitening wrap (201), showing the surface configured to contact the user's teeth and oral tissues during application. The whitening composition (203) is disposed across at least a portion of the interior surface and may be applied as a continuous layer or as discrete formulation zones (205) to facilitate controlled release. The central region (207) aligns with the anterior dentition, while lateral segments (209, 211) extend to engage posterior regions. The interior surface may optionally include microtexturing (213) or embossed features that enhance saliva absorption and facilitate conformal adhesion. The formulation layer may be visually distinct in appearance, color, or texture from the underlying substrate to indicate the active side. In multilayer configurations, a composition-carrying layer may be laminated to a structural backing to maintain integrity during storage and activation. F

What is claimed is:

1. An oral whitening wrap system comprising:
a. a conformable, biocompatible substrate configured to wrap around one or more dental arches; and
a whitening composition disposed on at least one surface of the substrate, the composition comprising:
(i) papain;
(ii) bromelain;
(iii) coconut oil;
(iv) aloe vera extract; and
(v) green tea extract;
wherein the substrate is free of synthetic adhesives and is configured to adhere to the teeth via saliva-activated hydration.

2. The oral whitening wrap system of claim 1, wherein the substrate comprises one or more hydrophilic polymers selected from the group consisting of hydroxypropyl cellulose, polyvinyl alcohol, and hydroxyethyl cellulose.

3. The oral whitening wrap system of claim 1, wherein the wrap is configured to conform to both labial and lingual surfaces of the teeth simultaneously.

4. The oral whitening wrap system of claim 1, wherein the papain and bromelain are present in a combined concentration of about 0.1% to 2.5% by weight.

5. The oral whitening wrap system of claim 1, wherein the composition is substantially free of hydrogen peroxide, carbamide peroxide, or other oxidative bleaching agents.

6. The oral whitening wrap system of claim 1, wherein the wrap further comprises a barrier layer laminated to the substrate to prevent premature exposure of the composition to air or moisture.

7. The oral whitening wrap system of claim 1, wherein the substrate includes pre-folded creases, ergonomic tapering, or anatomical curvature to facilitate fit along dental arches.

8. The oral whitening wrap system of claim 1, wherein the whitening composition includes a buffering agent selected from sodium citrate or potassium bicarbonate to maintain a pH between 6.0 and 7.5.

9. A method of whitening teeth comprising:
a. providing the oral whitening wrap system of claim 1;
b. placing the wrap around a dental arch of a user such that the whitening composition contacts tooth surfaces;
c. allowing the wrap to remain in place for a treatment period of 10 to 45 minutes; and
d. removing the wrap without rinsing, brushing, or adhesive residue,
wherein the whitening composition removes extrinsic stains without causing enamel damage or tooth sensitivity.

10. The method of claim 9, wherein step (b) comprises wrapping both upper and lower dental arches simultaneously using a dual-arch wrap configuration.

11. The method of claim 9, wherein the saliva of the user activates the adhesion of the wrap to the dental surfaces by hydrating the polymeric substrate.
